(19) United States
(12) Patent Application Publication
(10) Pub. No.: US 2026/0146531 A1
(43) Pub. Date: May 28, 2026

(54) METHOD FOR LOG PREDICTION FROM DRILLING CUTTINGS

(71) Applicant: Schlumberger Technology Corporation, Sugar Land, TX (US)

(72) Inventors: Simone Di Santo, Dammam (SA); Tetsushi Yamada, Cambridge, MA (US)

(21) Appl. No.: 19/387,223

(22) Filed: Nov. 12, 2025

Related U.S. Application Data
(60) Provisional application No. 63/723,934, filed on Nov. 22, 2024.

(51) Int. Cl. E21B 49/00; G01N 21/64; G01N 33/24; G01V 5/04; G06V 10/143; G06V 10/26; G06V 10/44; G06V 10/764; G06V 10/774; G06V 10/82; G06V 20/60

(52) U.S. Cl. CPC E21B 49/005; G01N 21/6456; G01N 33/24; G01V 5/04; G06V 10/143; G06V 10/26; G06V 10/44; G06V 10/764; G06V 10/774; G06V 10/82; G06V 20/60; E21B 2200/20; E21B 2200/22

(57) ABSTRACT

The present disclosure provides a method for predicting a target log frequency distribution within a depth interval. The method includes extracting drilling cuttings from a reference well and capturing a plurality of photographs of the drilling cuttings, wherein each photograph corresponds to a specific depth interval of a plurality of depth intervals. The method includes obtaining a reference target log from the reference well covering the plurality of depth intervals and extracting a plurality of image features from each photograph. The method includes capturing property variability within each depth interval including converting the plurality of image features and the reference target log into normalized frequency distributions. The method includes predicting model property variability by training a prediction model using the normalized frequency distributions of the image features as input data and the normalized frequency distribution of the reference target log as output data.

### CROSS-REFERENCE TO RELATED APPLICATIONS

This application claims priority to U.S. Provisional Application No. 63/723,934, filed 22 Nov. 2024, which is hereby incorporated by reference in its entirety.

### BACKGROUND

During drilling operations in oil and gas exploration, rock particles known as drilling cuttings are continuously generated as the drill bit penetrates subsurface formations and are transported to the surface by circulating drilling fluid. These cuttings provide valuable information about the geological formations encountered during drilling, including formation properties such as porosity, mineralogy, and grain density. Traditionally, the analysis of drilling cuttings has been performed manually by mud loggers who visually examine the rock particles to determine lithology and estimate formation characteristics. However, this manual process is time-consuming, subjective, and dependent on the experience and expertise of individual interpreters. The industry has recognized the potential for automating cuttings analysis to improve consistency, reduce interpretation time, and provide more quantitative geological information for real-time drilling decisions. While various approaches have been developed for automated lithology classification and rock property estimation, there remains a need for methods that can predict the distribution and variability of formation properties within the depth intervals represented by cuttings samples, rather than providing only average values that may not fully characterize the heterogeneity of subsurface formations.

### SUMMARY

According to an aspect of the present disclosure, a method for predicting a target log frequency distribution within a depth interval is provided. The method includes extracting drilling cuttings from a reference well. The method includes capturing a plurality of photographs of the drilling cuttings, wherein each photograph of the plurality of photographs corresponds to a specific depth interval of a plurality of depth intervals. The method includes obtaining a reference target log from the reference well covering the plurality of depth intervals. The method includes extracting a plurality of image features from each photograph. The method includes capturing property variability within each depth interval including converting the plurality of image features and the reference target log into normalized frequency distributions. The method includes predicting model property variability by training a prediction model using the normalized frequency distributions of the image features as input data and the normalized frequency distribution of the reference target log as output data.

According to other aspects of the present disclosure, the method may include one or more of the following features. The plurality of photographs may include white-light illuminated photographs of the drilling cuttings. The method may further include obtaining ultraviolet illuminated photographs of the drilling cuttings from the reference well and the target well. The method may further include obtaining auxiliary well logs from the reference well and the target well, wherein the auxiliary well logs are used to train the prediction model. The auxiliary well logs may include gamma-ray logs. Extracting the plurality of image features may include segmenting the plurality of photographs to identify individual rock particle features using a residual neural network autoencoder method. The segmenting the plurality of photographs may further identify unconsolidated material and consolidated rock particles in the plurality of photographs, and the prediction model may include a first prediction model for the unconsolidated material and a second prediction model for the unconsolidated material.

According to another aspect of the present disclosure, a system for predicting formation property variability from drilling cuttings is provided. The system includes an image acquisition device configured to capture photographs of drilling cuttings samples from wellbore operations. The system includes a processor electrically coupled to the image acquisition device. The system includes a non-transitory computer-readable medium storing instructions that, when executed by a processor, perform a prediction process. The prediction process includes segmenting the photographs to identify consolidated rock particles and unconsolidated materials. The prediction process includes extracting a plurality of features from the consolidated rock particles and unconsolidated materials. The prediction process includes converting the extracted features into normalized frequency distribution histograms that capture property variability within cutting sample intervals. The prediction process includes training, using reference well data, a first prediction model for target consolidated rock particles and a second prediction model for target unconsolidated loose fraction materials, wherein the first prediction model and the second prediction model are configured to predict property variability distributions within a target well. The prediction process includes applying the first prediction model and the second prediction model to predict formation property frequency distributions that characterize target property variability for target well.

According to other aspects of the present disclosure, the system may include one or more of the following features. The image acquisition device may be configured to capture white-light illuminated photographs and ultraviolet illuminated photographs of the drilling cuttings samples. The ultraviolet illuminated photographs may include fluorescence information for carbonate mineral identification. The processor may be further configured to obtain auxiliary well logs and incorporate the auxiliary well logs into the training of the first prediction model and the second prediction model and the prediction of the formation property frequency distributions. The auxiliary well logs may include gamma-ray logs. Extracting the plurality of features may include using a residual neural network autoencoder method.

According to another aspect of the present disclosure, a method for automated formation evaluation is provided. The method includes producing drilling cuttings from a reference wellbore. The method includes capturing a photograph of a sample of the drilling cuttings. The method includes analyzing the sample using object-based image analysis on the photograph to segment individual cutting instances and unconsolidated patches. The method includes extracting first image features from each individual cutting instance of the individual cutting instances. The method includes extracting second image features from each unconsolidated patch of the unconsolidated patches using a feature extraction algorithm. The method includes gathering feature variability information from the sample by generating frequency distribution histograms for each of the first image features and the second image features. The method includes consolidating the frequency distribution histograms to create input datasets corresponding to specific depth intervals within a target well that preserve the feature variability information. The method includes training a machine learning model using the input datasets and the frequency distribution histograms. The method includes predicting formation property frequency distributions that characterize property variability within cutting sample intervals for new wells by applying the trained machine learning model to consolidated input datasets derived from drilling cuttings photographs of the new wells.

According to other aspects of the present disclosure, the method may include one or more of the following features. The feature extraction algorithm may include a residual neural network autoencoder method. The photograph may be a white-light illuminated photograph, and the method may further include acquiring an ultraviolet illuminated photograph of the sample. The ultraviolet illuminated photograph may include fluorescence information for identifying carbonate minerals within the sample. The method may further include obtaining auxiliary well logs from the reference well and incorporating the auxiliary well logs into the training of the machine learning model. The auxiliary well logs may include gamma-ray logs. The formation property frequency distributions may include porosity frequency distributions that characterize porosity variability within the cutting sample intervals, and the method may further include deriving statistical parameters from each predicted porosity frequency distribution of the porosity frequency distributions to quantify property variability, the statistical parameters including mean, standard deviation, and distribution shape.

### BRIEF DESCRIPTION OF FIGURES

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example embodiments, the embodiments will be described and explained with additional specificity and detail through the use of the accompanying drawings in which:

FIG. 1 shows a wellsite system for drilling operations, according to aspects of the present disclosure;

FIG. 2 shows a schematic of relationships between formation properties and visual characteristics of rock, according to aspects of the present disclosure;

FIG. 3 shows a cuttings object image analysis workflow diagram, according to aspects of the present disclosure;

FIG. 4 shows a diagram showing integration of the method within an object-based image analysis workflow, according to aspects of the present disclosure;

FIG. 5 shows a schematic representation of model building and well log prediction stages, according to aspects of the present disclosure;

FIG. 6 shows a detailed workflow of the method with consolidated and unconsolidated processing paths, according to aspects of the present disclosure;

FIG. 7 shows scatter plots showing mean porosity prediction results, according to aspects of the present disclosure;

FIG. 8 shows mean porosity prediction results comparing consolidated and unconsolidated model predictions, according to aspects of the present disclosure; and

FIG. 9 shows predicted porosity frequency distribution histograms across different sample intervals, according to aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to methods and systems for predicting formation properties from drilling cuttings using digital image analysis and machine learning techniques. During drilling operations, rock particles crushed by a drill bit may be brought to the surface and analyzed to determine subsurface formation characteristics. Traditional analysis methods may rely on manual inspection by mud loggers, which can be time-consuming and subjective.

The disclosed methods may utilize digital photographs of drilling cuttings to predict formation properties such as porosity, grain density, and mineral composition. The approach may be based on the relationship between formation properties and macroscopic visual characteristics of rock particles, including spectral and texture variations that can be captured through digital imaging.

The methods may employ frequency distribution analysis to capture property variability within specific depth intervals rather than relying solely on average values. This approach may provide more detailed information about formation heterogeneity and property distributions within cutting sample intervals. The frequency distribution approach may enable the detection of bimodal or multimodal property distributions that could indicate the presence of multiple rock types or varying formation characteristics within a single depth interval.

The disclosed systems may include machine learning models trained using reference well data to establish correlations between image features and formation properties. The models may be configured to handle both consolidated rock particles and unconsolidated materials that may be present in drilling cuttings samples. Separate prediction models may be developed for consolidated and unconsolidated fractions, with results consolidated to provide comprehensive formation property predictions.

The methods may incorporate multiple types of input data, including white-light illuminated photographs, ultraviolet illuminated photographs, and auxiliary well logs such as gamma-ray measurements. The flexibility in input data types may allow the methods to be adapted to different drilling conditions and data availability scenarios. The ultraviolet illuminated photographs may provide additional information about mineral fluorescence characteristics that can enhance formation property predictions.

The disclosed approach may utilize object-based image analysis workflows that segment individual rock particles and extract quantitative features from each particle. Feature extraction may be performed using advanced techniques such as residual neural network autoencoder methods to capture detailed spectral and texture information from the rock particles. The extracted features may then be converted into normalized frequency distributions that preserve information about property variability within each sample interval.

During or after the analysis described, the near-real-time information gathered from the analysis an inform one or more changes in drilling operations. For example, the analysis and resulting information may be used to advise regarding changes in reservoir quality that enable informed drilling decisions even when logging while drilling is limited or absent. Also, for example, the methods and analysis described herein may collect continuous data that are normally available only in lab single-point measurements, for example grain size distribution. Another technical advantage of the methods and systems described herein includes post-mortem applications where cuttings archives can be used to extract the information when no log was performed, and a well was already drilled.

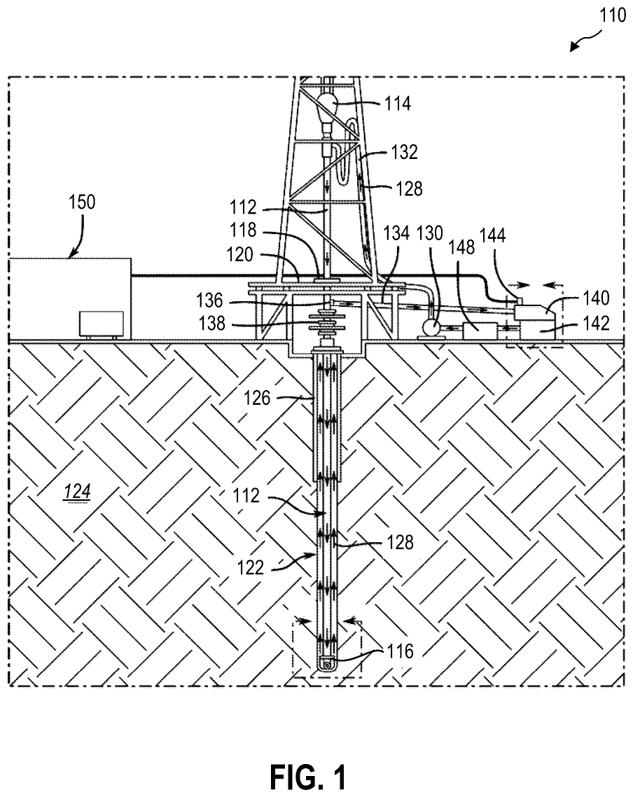

Referring to FIG. 1, a drilling operation 110 may be configured to extract samples from subterranean formations for geological analysis. The drilling operation 110 may include a drill string 112 that extends downward into a borehole 122 formed in an earth formation 124. A traveling block 114 may be positioned above the drill string 112 to facilitate movement of drilling equipment during operations.

A drill bit 116 may be located at the bottom end of the drill string 112 to cut through the earth formation 124 and create the borehole 122. The drill bit 116 may rotate to crush rock material, generating drilling cuttings that contain geological information about the earth formation 124. A rotary table 118 may be positioned on a driller floor 120 to provide rotational motion to the drill string 112 and the drill bit 116.

The drilling operation 110 may include a casing 126 that lines portions of the borehole 122 to provide structural support and prevent collapse of the borehole walls. Drilling fluid 128 may be circulated through the system to facilitate drilling operations and transport drilling cuttings to the surface. A mud pump 130 may be connected to the system via a mud line 132 to circulate the drilling fluid 128 downward through the drill string 112.

The drilling fluid 128 may flow back to the surface through a return flow line 134 after reaching the bottom of the borehole 122. The return flow line 134 may carry the drilling fluid 128 along with drilling cuttings that have been generated by the drill bit 116. A bell nipple 136 may be positioned at the top of the borehole 122 to direct the returning drilling fluid 128 and cuttings into the return flow line 134.

A blowout preventer 138 may be installed to provide safety control during drilling operations by preventing uncontrolled release of formation fluids. The drilling fluid 128 and cuttings may be directed to a shale shaker 140 that separates the drilling cuttings from the drilling fluid 128. The shale shaker 140 may use vibrating screens to separate solid particles from the liquid drilling fluid 128.

A shaker pit 142 may be positioned below the shale shaker 140 to collect the separated drilling cuttings for further processing and analysis. The drilling fluid 128 may be processed through additional equipment including a gas trap 144 that removes gas from the drilling fluid 128 before the fluid is stored in a mud pit 148 for reuse in the drilling operation 110.

A control system 150 may monitor and control various aspects of the drilling operation 110, including the circulation of drilling fluid 128 and the collection of drilling cuttings. The control system 150 may include one or more processors and non-transitory, computer-readable medium storing instructions, that when executed by the one or more processors, carries out the methods described herein. The one or more processors may be electrically coupled to various components of the systems described herein, including drilling equipment, image acquisition devices, and so forth. The drilling cuttings collected from the shaker pit 142 may contain both consolidated rock particles and unconsolidated material that can provide geological information about the earth formation 124. These drilling cuttings may be prepared for analysis using the methods described herein to extract comprehensive geological data from both consolidated and unconsolidated fractions of the sample.

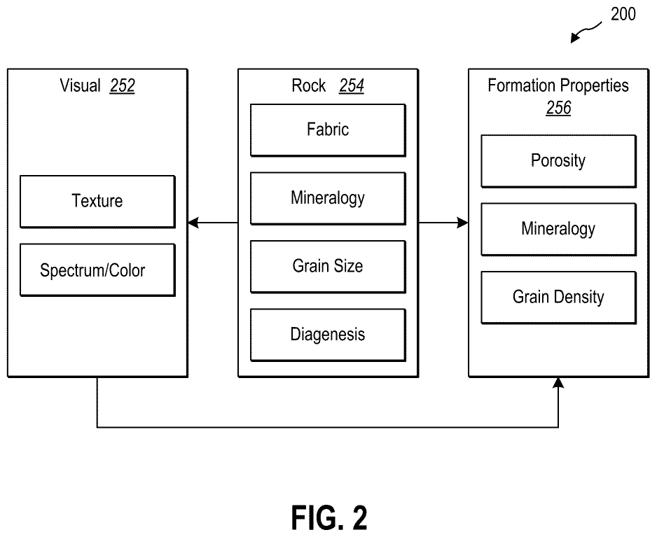

Referring to FIG. 2, a workflow 200 may illustrate the relationship between formation properties and visual macroscopic spectral and texture characteristics of rock particles. The disclosed methods may be based on the concept that lithology property variations may cause macroscopical variations in spectral characteristics and texture that can be quantified through captured digital photographs. The workflow 200 may demonstrate how formation properties correlate with observable visual characteristics that can be extracted from drilling cuttings images.

The workflow 200 may include a block 252 that represents visual characteristics including texture and spectral parameters that can be observed in drilling cuttings photographs. These visual characteristics may be directly measurable from digital images and may serve as input parameters for formation property prediction models. A block 254 may represent rock properties including fabric, mineralogy, grain size, and diagenesis parameters that influence the macroscopic appearance of rock particles.

A block 256 may represent formation properties including porosity, mineralogy, and grain density that may be predicted using the disclosed methods. The workflow 200 may show interconnected relationships between these different parameter categories through directional arrows that indicate how visual characteristics relate to rock properties and formation properties.

Porosity changes within rock formations may cause texture variations that can be detected through digital image analysis of drilling cuttings. Higher porosity rocks may exhibit different surface textures compared to lower porosity rocks due to differences in pore structure and grain arrangement. These texture variations may be quantified using image processing techniques that extract numerical features representing surface roughness, grain boundary characteristics, and pore visibility.

Clay content changes in clastic formations may result in spectral variations that typically appear as darker spectral ranges (coloration) in drilling cuttings photographs. Clay minerals may absorb more light compared to other mineral components, leading to observable spectral differences that can be measured through digital image analysis. The spectral variations may be quantified using spectral space analysis techniques that extract numerical values representing hue, saturation, and brightness characteristics.

Mineralogical changes in clastic formations may affect both spectral and texture variations in drilling cuttings samples. Different mineral compositions may exhibit distinct optical properties that influence both the spectral appearance and surface texture characteristics of rock particles. These combined spectral and texture variations may provide comprehensive information for predicting formation properties through machine learning models.

The relationships between formation properties and visual characteristics may be formation-specific, requiring different prediction models to be trained for each target formation. Geological formations with different depositional environments, diagenetic histories, and structural characteristics may exhibit unique relationships between visual appearance and formation properties. The formation-specific nature of these relationships may necessitate the development of customized prediction models for different geological settings.

The disclosed methods may be applied to rock particles that have been cleaned and prepared for digital photography. The preparation process may reveal rock texture and spectral characteristics by removing drilling fluids and other materials that could mask or cover the natural appearance of the rock particles. The cleaning and preparation methods may enhance the visibility of formation-related visual characteristics while maintaining the integrity of the rock particle surfaces.

The workflow 200 may, but does not necessarily in all examples, enable prediction of multiple formation properties including total porosity, effective porosity, rock mineral composition, and grain density. Total porosity predictions may provide information about the overall pore space within rock formations, while effective porosity predictions may focus on interconnected pore networks that contribute to fluid flow characteristics. Rock mineral composition predictions may support geological characterization efforts by identifying the relative abundance of different mineral phases within the formation.

Grain density predictions may provide formation evaluation data that supports reservoir characterization and completion design decisions. The grain density information may be combined with porosity predictions to calculate bulk density values and other petrophysical parameters. The multiple target log prediction capabilities may allow comprehensive formation evaluation using drilling cuttings as the primary data source when conventional logging tools may not be available or practical to deploy.

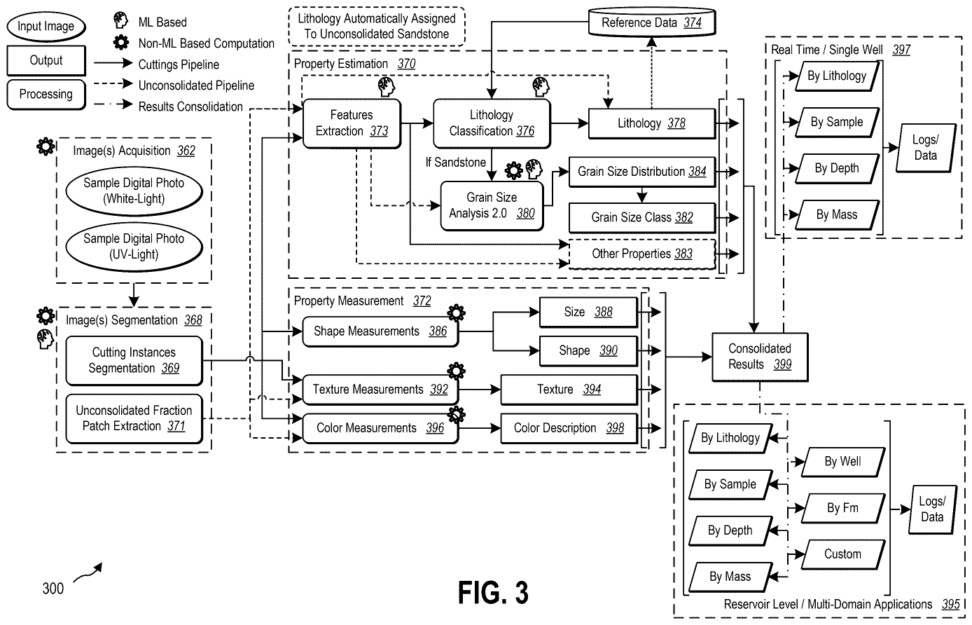

Referring to FIG. 3, a workflow 300 may illustrate a cuttings object-based image analysis (OBIA) workflow that forms the foundation for the disclosed formation property prediction methods. The workflow 300 may provide a comprehensive framework for processing drilling cuttings samples through automated image analysis techniques. The workflow 300 may be configured to handle both consolidated rock particles and unconsolidated materials through separate processing pipelines that accommodate different material characteristics.

The workflow 300 may include an acquisition workflow 362 that encompasses the initial stages of sample collection and preparation for digital imaging. The acquisition workflow 362 may begin with sample preparation steps that prepare drilling cuttings for optimal image capture and analysis. Sample preparation may include drying the drilling cuttings in an oven to remove moisture and drilling fluids that could interfere with image quality or mask natural rock characteristics.

The acquisition workflow 362 may include sieving operations using meshes with sizes to select particles that fall within a specific size range. The sieving process may separate drilling cuttings into size fractions that are suitable for digital imaging and automated analysis. The size selection may enhance the consistency of image analysis results by providing particles with similar dimensional characteristics for feature extraction processes.

The workflow 300 may include multiple computational components that process both consolidated rock particles and unconsolidated material to obtain comprehensive geological data about subterranean formations. The workflow 300 may integrate various measurement techniques and analysis methods to characterize different properties of the drilling cuttings sample, each block of the workflow 300 shown in FIG. 3 may include an icon indicating a process step which may be carried out by non-machine learning based computation and those that may be carried out using machine-learning based computations. These blocks and indications of machine-learning steps are exemplary only and not meant to be limiting. Other embodiments of the workflow 300 may include other scenarios where machine-learning computations is or is not used.

The workflow 300 may include an acquisition workflow 362 that handles the initial processing of input data from the drilling cuttings sample. The acquisition workflow 362 may receive input from visible light digital photographs at block 364 and UV fluorescence digital photographs at block 366 that have been captured during the photograph acquisition process.

At block 368, the outlining instance segmentation may create precise delineations around each identified object from the photographs to enable accurate measurements and analysis of individual components within the sample. At block 368, the acquisition workflow 362 may complete the initial processing steps, including cutting instances segmentation for rock particles at block 369 and unconsolidated fraction (loose material) patch extraction from the photographs at block 371, and provide segmented data to subsequent analysis components within the workflow 300.

The workflow 300 may include a property estimation workflow 370 that processes the segmented data to estimate various geological properties of the sample materials. The property estimation workflow 370 may utilize both machine learning-based and non-machine learning-based computational approaches to analyze the characteristics of consolidated particles and unconsolidated material. The property estimation workflow 370 may generate quantitative estimates of geological properties that can be used to characterize the subterranean formation from which the sample was extracted.

A property measurement workflow 372 may operate in parallel with the property estimation workflow 370 to perform direct measurements on the segmented objects identified during the acquisition workflow 362. At block 374, reference data may be used as an input for the machine-learning computations within the property estimation workflow 370.

The property estimation workflow 370 may begin with feature extraction at block 373. At block 376, the property measurement workflow 372 may estimate lithology classifications of the sample and determine lithology at 378. Also, after lithology classification at block 376, grain size analysis of the sample can occur as shown at block 380. After grain size analysis at block 380, the grain size class can be estimated at block 382 and the grain size distribution can be estimated at block 384.

With reference to the property measurement workflow 372, after cutting instances segmentation at block 368, the sample can undergo shape measurements at block 386, texture measurements at block 392, and spectral measurements at block 396. After shape measurements at block 386, the size (at block 388) and shape (at block 390) of the consolidated and unconsolidated material within the sample can be measured. After texture measurements at block 392, the texture can be determined at block 394. Likewise, after spectral measurements at block 396, the spectral description can be determined at block 398. The results from the property estimation workflow 370, including the lithology at block 378, the grain size class at block 382, and the grain size distribution at block 384, may be combined with the results from the property measurement workflow 372, including the size at block 388, the shape at block 390, the texture at block 394, and the spectral description at block 398, into consolidated results at block 399. Other properties may be estimated at block 383.

The consolidated results at block 399 of the workflow 300 may complete the analysis process by generating final results that characterize the geological properties of the subterranean formation. Real-time, single well outputs are shown at block 397 and may include outputs organized or categorized by lithology, sample, depth, and measurements together as LiOBIA logs and data. Multi-domain outputs are shown at block 395 and may include similar outputs as noted above at block 397, with the addition of outputs organized or categorized by well or other outputs together to form LiOBIA logs and data.

The workflow 300 may utilize both ML-based and non-ML based computational elements throughout the processing chain to ensure robust analysis of the drilling cuttings sample. The ML-based computations may employ machine learning algorithms to identify patterns and classify materials, while non-ML based computations may perform direct measurements and calculations based on established geological principles and reference data.

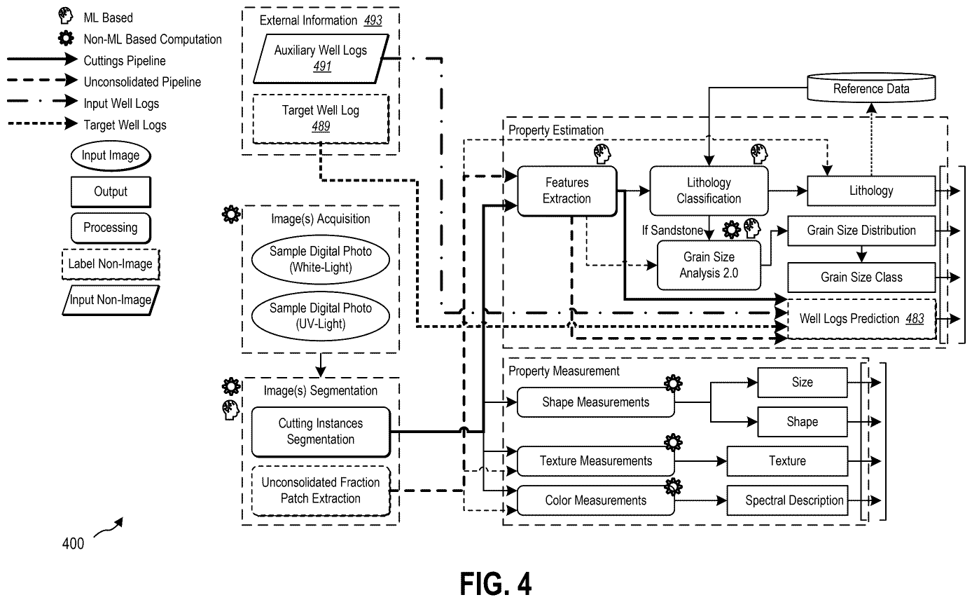

Referring to FIG. 4, a workflow 400 may illustrate how the disclosed well log prediction method integrates within the Object-Based Image Analysis (OBIA) workflow to expand the capabilities of automated lithology analysis. The workflow 400 may demonstrate the position of the well log prediction method in relation to existing OBIA components and may show how the method leverages established photographic acquisition standards, image feature extraction methods, and segmentation results to achieve formation property log prediction.

The workflow 400 may build upon the foundation established by the workflow 300 shown in FIG. 3 by incorporating additional processing steps that enable prediction of formation properties from drilling cuttings images. The workflow 400 may utilize the same photographic acquisition standards and segmentation techniques established in the workflow 300 while extending the analysis capabilities to include quantitative formation property predictions.

The workflow 400 may receive external information at block 493 that includes auxiliary well log data and target well log information to enhance the formation property prediction process. The auxiliary well log information at block 491 may include gamma-ray logs, neutron logs, density logs, and other conventional wireline measurements that provide geological context for the target formation intervals. These auxiliary logs may serve as supplementary input data that can improve the correlation between image features and formation properties by providing additional geological constraints and validation data.

Target well log information at block 489 may include existing log measurements from the target well that can be used to calibrate and validate the prediction models. The target well log data may encompass previously acquired wireline logs, logging-while-drilling measurements, or other formation evaluation data that characterizes the geological properties of the target formation. This target well information may enable the prediction models to account for well-specific geological conditions and improve prediction accuracy through local calibration.

The information from blocks 491 and 489 may be integrated at block 483 to generate enhanced formation property predictions that combine image-based analysis with conventional log data. Block 483 may represent the integration point where well log prediction capabilities are incorporated into the existing OBIA framework to produce comprehensive formation evaluation results. The auxiliary well log information from block 491 may be used to constrain the prediction models and provide geological context that enhances the interpretation of image features extracted from drilling cuttings.

The target well log information from block 489 may be processed through the same frequency distribution computation methods used for image features to maintain consistency in the machine learning model inputs. The frequency distributions of target well log data may be compared with predicted frequency distributions to validate model performance and adjust prediction parameters. This comparison process may enable real-time quality control and model refinement during drilling operations.

Block 483 may generate additional results from the well log prediction process that complement the grain size analysis, grain size distribution, and lithology results produced by the workflow 300. The well log prediction results may include porosity frequency distributions, grain density predictions, mineral composition estimates, and other formation properties that are derived from the integrated analysis of image features and auxiliary log data. These additional results may provide quantitative formation property information that extends beyond the qualitative lithology classifications traditionally obtained from drilling cuttings analysis.

The well log predictions generated at block 483 may be incorporated into the consolidated results shown at block 399 of workflow 300 to provide comprehensive formation evaluation data. The integration of well log predictions with traditional OBIA results may create a unified output that includes both lithological classifications and quantitative formation property logs. This consolidated approach may enable drilling teams to access both qualitative geological descriptions and quantitative petrophysical parameters from the same drilling cuttings sample analysis workflow.

The consolidated results at block 399 may include the well log predictions as part of the LiOBIA logs and data outputs, providing depth-based formation property logs that characterize property distributions within the formation intervals represented by the drilling cuttings samples. The well log prediction component may enhance the value of the consolidated results by providing formation property information that can support real-time drilling decisions, completion design, and reservoir characterization efforts.

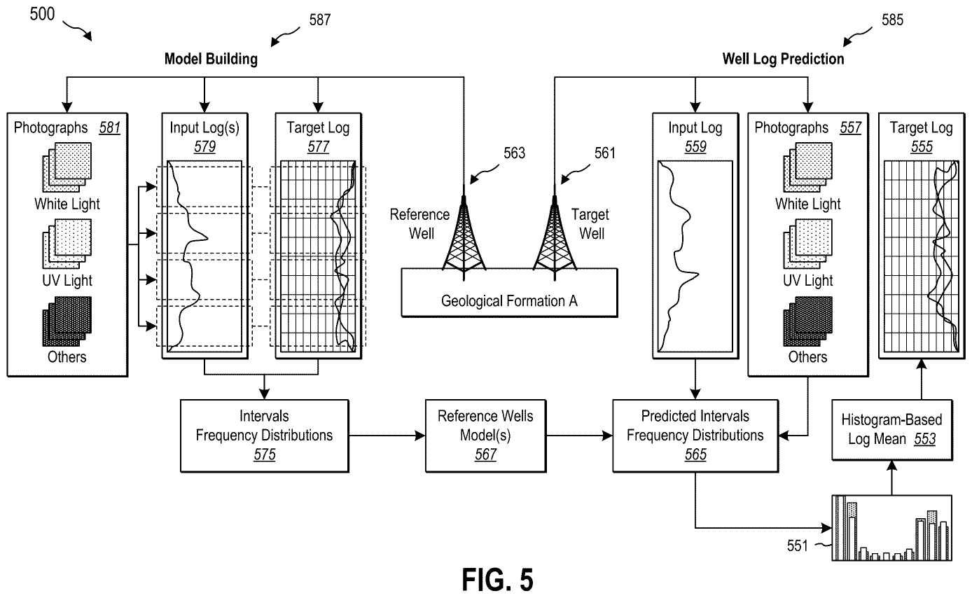

Referring to FIG. 5, a workflow 500 may illustrate a schematic representation of the two stages of the disclosed methods for predicting formation properties from drilling cuttings. The workflow 500 may include a model building workflow 587 and a well log prediction workflow 585 that operate to establish correlations between image features and formation properties, then apply these correlations to predict formation properties in target wells. The two-stage structure may enable the methods to be applied across multiple wells within a formation once a prediction model has been established using reference well data.

The model building workflow 587 may represent the first stage of the workflow 500, where a prediction model may be developed using data from a reference well 563. The model building workflow 587 may utilize multiple types of input data to establish robust correlations between drilling cuttings image characteristics and formation properties. The reference well 563 may provide the training data necessary to develop machine learning models that can predict formation properties from image features and auxiliary log data.

A block 581 may represent photographs that serve as primary input data for the model building workflow 587. The photographs at block 581 may include white-light and UV-light illuminated images of drilling cuttings samples that have been prepared and captured according to standardized protocols. The photographs may capture macroscopic visual characteristics including spectral and texture variations that correlate with formation properties such as porosity, grain density, and mineral composition.

A block 579 may represent input logs that provide auxiliary well log data for the model building workflow 587. The input logs at block 579 may include gamma-ray logs, neutron logs, density logs, or other conventional wireline measurements that characterize the geological properties of the reference well 563. These auxiliary logs may serve as supplementary input data that can enhance the correlation between image features and formation properties by providing additional geological context and constraints.

The target log at block 577 may include porosity measurements, grain density values, mineral composition data, or other formation properties that serve as the ground truth data for model training. The target log may cover the same depth intervals as the photographs at block 581 and the input logs at block 579 to enable proper correlation and model development.

A block 575 may represent intervals frequency distributions that may be created during the model building workflow 587. The intervals frequency distributions at block 575 may be generated by converting the image features extracted from the photographs at block 581, the auxiliary log data from block 579, and the target log data from block 577 into normalized frequency distribution histograms. These frequency distributions may capture property variability within specific depth intervals rather than relying solely on average values.

A block 567 may represent a reference well model that may be developed during the model building workflow 587. The reference well model at block 567 may be trained using the intervals frequency distributions at block 575 as input data and may be configured to predict formation property frequency distributions based on image features and auxiliary log data. The reference well model may utilize machine learning algorithms such as XGBoost, neural networks, or other regression techniques to establish correlations between input features and target formation properties.

The well log prediction workflow 585 may represent the second stage of the workflow 500, where the reference well model at block 567 may be applied to predict formation properties in a target well 561. The well log prediction workflow 585 may utilize the same types of input data as the model building workflow 587 but may not require target log measurements since these properties are being predicted rather than measured.

A block 557 may represent photographs from the target well 561 that serve as primary input data for the well log prediction workflow 585. The photographs at block 557 may be captured using the same standardized protocols and imaging conditions as the photographs at block 581 to ensure consistency in image quality and feature extraction results. The photographs may be processed through the same segmentation and feature extraction methods used during model training to generate comparable input data for the prediction process.

A block 559 may represent input logs from the target well 561 that provide auxiliary well log data for the well log prediction workflow 585. The input logs at block 559 may include the same types of auxiliary measurements as the input logs at block 579 to maintain consistency in the prediction model inputs. The auxiliary log data may be processed through the same frequency distribution computation methods used during model training to generate compatible input features for the prediction process.

A block 565 may represent predicted intervals frequency distributions that may be generated by applying the reference well model at block 567 to the input data from blocks 557 and 559. The predicted intervals frequency distributions at block 565 may characterize the expected variability of formation properties within each depth interval represented by the drilling cuttings samples from the target well 561. These predicted distributions may provide more detailed information about formation heterogeneity compared to single-value predictions.

A block 551 may represent histograms that may be generated from the predicted intervals frequency distributions at block 565. The histograms at block 551 may provide visual representations of the predicted formation property distributions and may enable identification of bimodal or multimodal distributions that could indicate the presence of multiple rock types or varying formation characteristics within individual depth intervals.

A block 553 may represent histogram-based log mean values that may be calculated from the histograms at block 551. The histogram-based log mean at block 553 may be computed by multiplying the frequency of each histogram bin by the bin center value and dividing the sum by the total number of bins. This calculation may provide mean formation property values that account for the full distribution of properties within each depth interval.

A block 555 may represent a target log that may be generated as the final output of the well log prediction workflow 585. The target log at block 555 may include predicted formation properties such as porosity, grain density, or mineral composition that have been derived from the drilling cuttings image analysis and auxiliary log data. The target log may provide depth-based formation property information that can support drilling decisions, completion design, and reservoir characterization efforts.

The workflow 500 may be designed to be flexible in terms of required data, allowing the input information to be increased if the complexity of the prediction requires additional constraints. Total porosity, effective porosity, rock mineral composition, and grain density may be examples of measurements that can be predicted using the disclosed methods. The variation of these properties within rock formations may produce changes in the macroscopic appearance of rock particles that can be captured through drilling cuttings photographs.

The photographs at blocks 581 and 557 may be integrated with other basic well logs such as gamma-ray measurements, surface logs such as gas chromatography data, or drilling measurement-while-drilling parameters such as rate of penetration and weight on bit to improve prediction accuracy. The input data may also be expanded by including other types of photographs of the same drilling cuttings samples, such as ultraviolet illuminated images, infrared images, multispectral images, or hyperspectral images that can provide additional information about mineral characteristics and formation properties.

The method for predicting a target log frequency distribution within a depth interval may include obtaining a reference target log from the reference well 563 covering a plurality of depth intervals that correspond to the drilling cuttings samples. The reference target log may provide the ground truth formation property measurements that enable the development of correlations between image features and formation properties during the model building workflow 587.

A system for predicting formation property variability from drilling cuttings may include the components and workflows illustrated in FIG. 5, including the capability to process both reference well data for model development and target well data for formation property prediction. The system may be configured to handle the two-stage workflow structure and may include processing capabilities for image analysis, frequency distribution computation, machine learning model training, and formation property prediction.

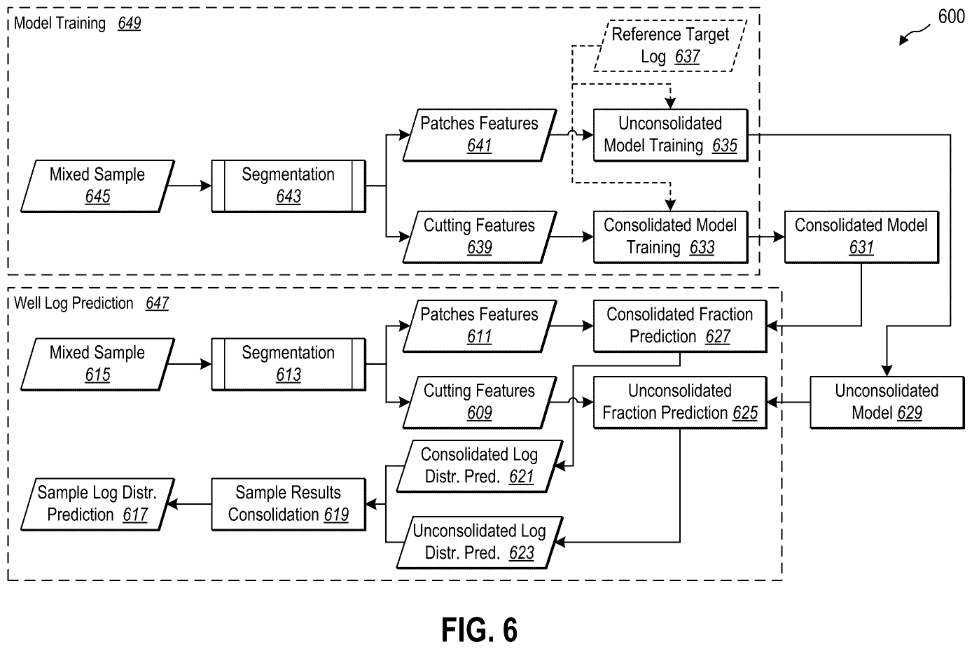

Referring to FIG. 6, a workflow 600 may illustrate the detailed flowchart for model building and prediction stages of the disclosed formation property prediction methods. The workflow 600 may include a model training workflow 649 and a well log prediction workflow 647 that operate sequentially to establish formation property prediction capabilities from drilling cuttings analysis. The workflow 600 may demonstrate how both consolidated rock particles and unconsolidated materials are processed through separate but parallel pathways to generate comprehensive formation property predictions.

The model training workflow 649 may represent the first stage of the workflow 600, where prediction models are developed using reference well data to establish correlations between drilling cuttings characteristics and formation properties. The model training workflow 649 may be subdivided into four steps: data acquisition and selection, data preparation, frequency distribution computation, and prediction model building. These steps may provide a systematic approach to developing robust prediction models that can accurately correlate image features with formation properties.

During the data acquisition and selection step, the input dataset may be identified, and decisions may be made regarding the use of auxiliary well logs and additional images such as ultraviolet illuminated photographs, infrared illuminated photographs, multispectral photographs, or hyperspectral photographs. The choice of input data types may depend largely on data availability during the prediction phase and the specific target log to be predicted. For example, in clastic environments where total porosity is the target log, ultraviolet illuminated photographs may provide information about carbonate cement due to natural fluorescence of carbonate minerals when exposed to ultraviolet light.

The workflow 600 may incorporate surface logs such as gas chromatography data or drilling measurement-while-drilling parameters such as rate of penetration and weight on bit as auxiliary input data to enhance prediction accuracy. The input data may also be expanded by including infrared illuminated photographs for enhanced rock characterization, multispectral photographs for advanced mineral identification, or hyperspectral photographs for detailed spectral analysis. All well logs and cuttings samples used may cover a common depth interval to ensure proper correlation during model training.

At block 645, mixed sample data may serve as the initial input for the model training workflow 649. The mixed sample data at block 645 may include drilling cuttings samples that contain both consolidated rock particles and unconsolidated materials that require separate processing pathways. The mixed sample data may be prepared according to standardized protocols that reveal rock texture and spectral characteristics while maintaining the integrity of both consolidated and unconsolidated fractions.

At block 643, a segmentation action may be performed on the mixed sample data to separate consolidated rock particles from unconsolidated materials. The segmentation at block 643 may utilize object-based image analysis techniques to identify individual cutting instances and unconsolidated patches within the drilling cuttings photographs. The segmentation process may employ machine learning algorithms to accurately distinguish between different material types based on visual characteristics such as shape, texture, and size.

At block 639, cutting features data may be generated from the consolidated rock particles identified during the segmentation at block 643. The cutting features at block 639 may represent quantitative characteristics extracted from individual rock particle instances using advanced image processing techniques. The feature extraction may utilize a residual neural network autoencoder method to extract 64 separate features for each image that capture detailed spectral and texture information from the consolidated rock particles.

At block 641, patches features data may be generated from the unconsolidated materials identified during the segmentation at block 643. The patches features at block 641 may represent quantitative characteristics extracted from unconsolidated patches using the same feature extraction algorithm employed for consolidated rock particles. The feature extraction algorithm may include a residual neural network autoencoder method that generates comparable feature sets for both consolidated and unconsolidated materials to enable consistent analysis across different material types.

During the data preparation step, the target log that may be used to label and train the prediction models may be split into subset depth intervals that match the depth intervals for each drilling cuttings sample. At block 637, a reference target log may serve as optional input to both the consolidated rock particle model training and the unconsolidated material model training processes. The reference target log at block 637 may provide ground truth formation property measurements that enable the development of correlations between image features and formation properties during model training.

Every drilling cuttings sample may match a specific well log interval, enabling image features and auxiliary well logs to be processed through the frequency distribution computation step. During the frequency distribution computation step, all computed image features, auxiliary well logs, and target logs may be converted to normalized frequency distributions with predefined bins ranges and size. The distribution bins may be set according to the data range and statistics to ensure optimal representation of property variability within each depth interval.

At block 633, consolidated rock particle model training may be performed using the cutting features data from block 639 and the reference target log from block 637. The consolidated rock particle model training at block 633 may utilize machine learning algorithms to establish correlations between image features extracted from consolidated rock particles and formation properties. The training process may employ different regression models including Linear Regression, Support Vector Regression, Ridge Regression, Lasso Regression, Decision Trees, Random Forest, and XGBoost algorithms.

At block 635, unconsolidated material model training may be performed using the patches features data from block 641 and the reference target log from block 637. The unconsolidated material model training at block 635 may develop separate prediction capabilities for unconsolidated materials that may exhibit different relationships between visual characteristics and formation properties compared to consolidated rock particles. The training process may utilize the same regression model options as the consolidated rock particle training but may be optimized specifically for unconsolidated material characteristics.

During the prediction model building step, the models may be built using data selected from reference wells after model validation. Model validation may consist of splitting the reference data into train and test subsets, where the split can be based on wells or on a general random split of all available reference intervals. The models may be trained on the train subset, and predictions may be performed on the test dataset where predicted and actual target log data can be compared to assess model prediction performance.

At block 631, a consolidated model may be generated as output from the consolidated rock particle model training at block 633. The consolidated model at block 631 may represent a trained machine learning model that can predict formation property frequency distributions based on image features extracted from consolidated rock particles. The consolidated model may incorporate hyperparameter optimization to build the final prediction model that provides optimal performance for consolidated rock particle analysis.

At block 629, an unconsolidated model may be generated as output from the unconsolidated material model training at block 635. The unconsolidated model at block 629 may represent a trained machine learning model that can predict formation property frequency distributions based on image features extracted from unconsolidated materials. The unconsolidated model may be optimized separately from the consolidated model to account for the different visual characteristics and formation property relationships exhibited by unconsolidated materials.

The well log prediction workflow 647 may represent the second stage of the workflow 600, where the trained models from the model training workflow 649 may be applied to predict formation properties in target wells. The well log prediction workflow 647 may include four steps: data acquisition and collection, data preparation, frequency distribution computation, and target log prediction. The first three steps may be similar to the model training workflow 649 with the difference that data selection tasks are not required and target well logs are not present since formation properties are being predicted rather than measured.

At block 615, mixed sample data from a target well may serve as input for the well log prediction workflow 647. The mixed sample data at block 615 may include drilling cuttings samples from the target well that contain both consolidated rock particles and unconsolidated materials requiring the same processing pathways established during model training. The mixed sample data may be prepared using the same standardized protocols employed during the model training workflow 649 to ensure consistency in sample preparation and image quality.

At block 613, a segmentation action may be performed on the mixed sample data from block 615 to separate consolidated rock particles from unconsolidated materials in the target well samples. The segmentation at block 613 may utilize the same object-based image analysis techniques employed during the model training workflow 649 to ensure consistent identification of consolidated and unconsolidated fractions. The segmentation process may maintain the same accuracy and reliability standards established during model development.

At block 611, cutting features data may be generated from the consolidated rock particles identified during the segmentation at block 613. The cutting features at block 611 may be extracted using the same residual neural network autoencoder method employed during model training to ensure compatibility with the consolidated model at block 631. The feature extraction process may generate the same 64 separate features for each image that were used during model training to maintain consistency in input data characteristics.

At block 609, patches features data may be generated from the unconsolidated materials identified during the segmentation at block 613. The patches features at block 609 may be extracted using the same feature extraction algorithm employed during model training to ensure compatibility with the unconsolidated model at block 629. The feature extraction process may generate comparable feature sets that enable accurate prediction of formation properties from unconsolidated materials in the target well.

At block 627, consolidated fraction prediction may be performed using the cutting features data from block 611 and the consolidated model from block 631. The consolidated fraction prediction at block 627 may generate formation property frequency distributions for consolidated rock particles in the target well samples. The prediction process may utilize the trained consolidated model to correlate image features with formation properties based on the relationships established during model training.

At block 625, unconsolidated fraction prediction may be performed using the patches features data from block 609 and the unconsolidated model from block 629. The unconsolidated fraction prediction at block 625 may generate formation property frequency distributions for unconsolidated materials in the target well samples. The prediction process may utilize the trained unconsolidated model to establish correlations between image features and formation properties for unconsolidated materials.

At block 621, consolidated log distribution prediction may be generated as output from the consolidated fraction prediction at block 627. The consolidated log distribution prediction at block 621 may represent predicted formation property frequency distributions that characterize property variability within consolidated rock particles for each depth interval. These frequency distributions may capture the full range of formation property values rather than single average values, providing detailed information about formation heterogeneity.

At block 623, unconsolidated log distribution prediction may be generated as output from the unconsolidated fraction prediction at block 625. The unconsolidated log distribution prediction at block 623 may represent predicted formation property frequency distributions that characterize property variability within unconsolidated materials for each depth interval. These frequency distributions may provide complementary information to the consolidated predictions and may be particularly valuable for formations with significant unconsolidated fractions.

At block 619, sample results consolidation may be performed by combining the consolidated log distribution prediction from block 621 and the unconsolidated log distribution prediction from block 623. The sample results consolidation at block 619 may statistically integrate the separate predictions from consolidated and unconsolidated fractions to generate unified formation property predictions for each drilling cuttings sample. The consolidation process may account for the relative proportions of consolidated and unconsolidated materials within each sample to ensure accurate representation of overall formation properties.

At block 617, sample log distribution prediction may be generated as the final output from the sample results consolidation at block 619. The sample log distribution prediction at block 617 may represent comprehensive formation property frequency distributions that characterize property variability within each depth interval represented by the drilling cuttings samples. These predicted distributions may provide detailed information about formation heterogeneity and may enable identification of bimodal or multimodal property distributions that could indicate the presence of multiple rock types or varying formation characteristics within individual depth intervals.

The frequency distribution computation methods employed in both the model training workflow 649 and the well log prediction workflow 647 may convert extracted features into normalized frequency distribution histograms that capture property variability within cutting sample intervals. The normalized frequency distributions may preserve information about the full range of property values within each depth interval rather than reducing the data to single average values. This approach may enable the detection of formation heterogeneity and property distributions that could be masked by traditional averaging methods.

The workflow 600 may demonstrate how the presence of unconsolidated or loose fraction in drilling cuttings samples may be automatically handled through the segmentation process, with unconsolidated materials passing through a different training model compared to consolidated rock particles. The prediction workflow may account for this separation by predicting frequency distributions separately using respective models for consolidated rock particles and unconsolidated materials, with results eventually statistically consolidated for each sample to provide comprehensive formation property predictions.

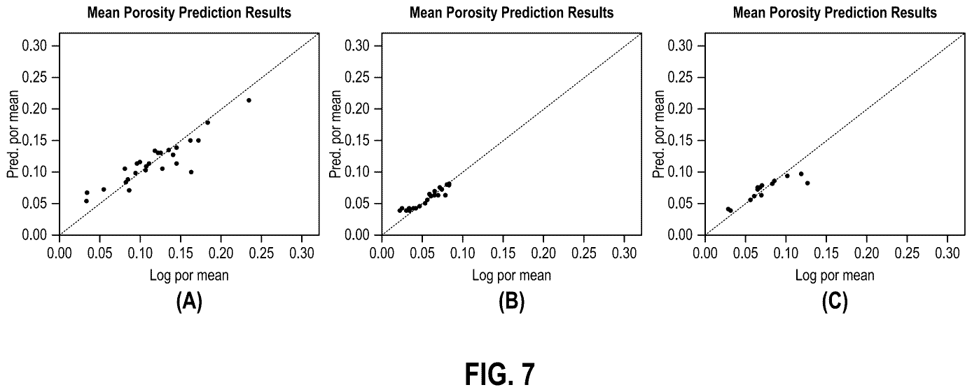

Referring to FIG. 7, the prediction model validation process may include splitting reference well data into training and testing subsets to conduct blind testing of model performance and assess prediction accuracy. The validation process may utilize either well-based splitting, where entire wells are designated as training or testing datasets, or random splitting, where reference intervals are randomly distributed between training and testing subsets. This validation approach may enable assessment of model performance on data that was not used during the training process, providing an unbiased evaluation of the model's ability to predict formation properties from drilling cuttings image features.

The validation process may generate predicted mean target log values that are computed from predicted distribution histograms and compared with corresponding values computed from reference target logs during the assessment phase. FIG. 7 illustrates three crossplot examples showing the relationship between predicted mean porosity values and reference mean porosity values, demonstrating varying levels of prediction accuracy across different scenarios. Plot (A) may show the worst performance scenario with a root mean square error (RMSE) of 2 porosity units, plot (B) may demonstrate the best prediction performance with an RMSE of 0.8 porosity units, and plot (C) may illustrate good performance with some decrease in accuracy for higher porosity values, resulting in an RMSE of 1.4 porosity units.

The performance assessment may utilize statistical metrics including mean error, residuals, and RMSE calculations to quantify model prediction accuracy and identify potential performance variations across different formation property ranges. The validation results may indicate whether the model performs consistently across all property value ranges or exhibits reduced accuracy in specific intervals, such as the decreased performance at higher porosity values shown in plot (C) of FIG. 7. This performance assessment approach may enable model refinement and optimization by identifying areas where prediction accuracy can be improved through additional training data, feature engineering, or model parameter adjustments.

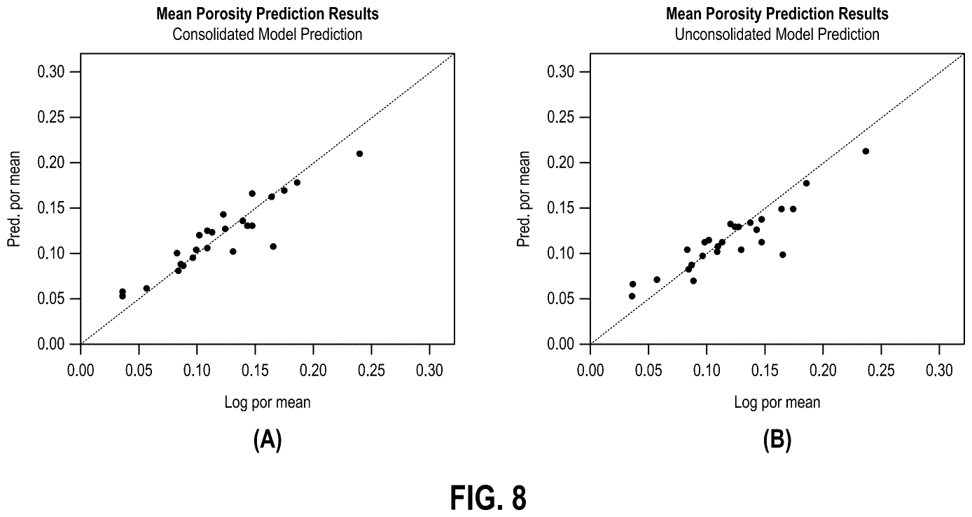

Referring to FIG. 8, the disclosed methods may handle both consolidated rock particles and unconsolidated loose materials through separate prediction models that are trained specifically for each fraction type. The segmentation process may automatically identify consolidated rock particles and unconsolidated materials within drilling cuttings samples, enabling each fraction to be processed through dedicated analysis pathways. FIG. 8 illustrates histogram distribution mean reference versus predicted plots for the same well, where plot (A) shows consolidated model prediction results and plot (B) shows unconsolidated model prediction results, demonstrating that prediction accuracy may be comparable between both fraction types with the unconsolidated prediction being slightly less accurate than the consolidated prediction.

The separate prediction models may be developed during the model training workflow where consolidated rock particles and unconsolidated materials are processed through parallel pathways that utilize the same feature extraction algorithms but generate distinct correlations between image features and formation properties. A processor electrically coupled to an image acquisition device may execute the segmentation algorithms that distinguish between consolidated and unconsolidated fractions based on visual characteristics such as particle size, shape regularity, and texture patterns. The processor may then apply separate feature extraction processes to generate cutting features data for consolidated particles and patches features data for unconsolidated materials, enabling each fraction type to be characterized using appropriate analytical approaches.

During the prediction phase, the method may apply a first prediction model for consolidated rock particles and a second prediction model for unconsolidated materials to predict formation property frequency distributions that characterize target property variability for a target well. The separate predictions may be statistically consolidated through a sample results consolidation process that accounts for the relative proportions of consolidated and unconsolidated materials within each drilling cuttings sample. This consolidation approach may enable the method to generate comprehensive formation property predictions for samples containing mixed materials while maintaining the accuracy advantages of fraction-specific modeling approaches, as demonstrated by the comparable prediction performance shown in both plots of FIG. 8.

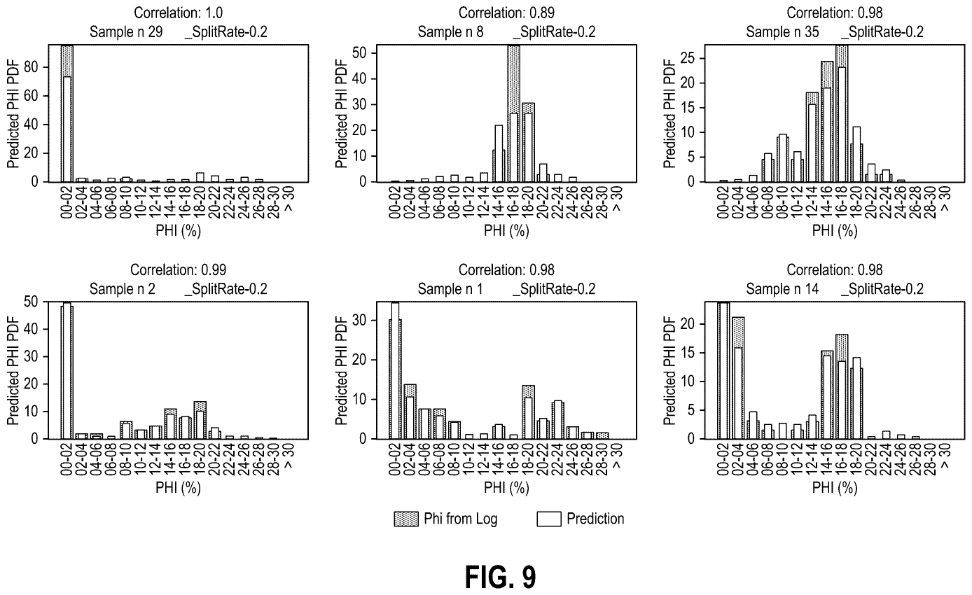

Referring to FIG. 9, the disclosed methods may generate predicted frequency distribution histograms that demonstrate the capability to capture porosity distribution variability within depth intervals represented by drilling cuttings samples. The predicted frequency distribution histograms may provide detailed characterization of formation property variability that extends beyond single-value predictions by preserving information about the full range of property values within each cutting sample interval. The frequency distribution approach may enable identification of complex property distributions including bimodal or multimodal patterns that could indicate the presence of multiple rock types or varying formation characteristics within individual depth intervals.

The predicted frequency distribution histograms shown in FIG. 9 may illustrate examples of porosity distribution predictions generated using white-light illuminated photographs and gamma-ray log data as input to predict total porosity as a target log in clastic formations. The histograms may be composed of 15 bins of 2 porosity units each, providing a total range of 30 porosity units for comprehensive characterization of porosity variability within the formation intervals. An XGBoost machine learning model may be utilized to generate the predictions that capture both unimodal and bimodal porosity distributions across different depth intervals.

The upper row of histograms in FIG. 9 may illustrate intervals with unimodal distribution patterns where a highest peak may be similar to an interval average, indicating relatively uniform porosity characteristics within those depth intervals. These unimodal distributions may represent formation intervals with consistent rock properties and relatively homogeneous porosity characteristics. The predicted distribution histograms may accurately capture the central tendency and variability of porosity values within these uniform intervals.

The lower row of histograms in FIG. 9 may demonstrate the capability of the disclosed methods to capture bimodal porosity distributions where a computed mean may not be correlated to the potential of the interval. The lower left histogram may show an example where most of the interval exhibits porosity values below 2 porosity units, but layers with porosity values measured at 20 porosity units may also be present within the same depth interval. The highest peak of porosity in a high value region may indicate that very porous layers are present in the predicted interval regardless of other layers in the same interval showing very low porosity values.

The bimodal distribution capability may be particularly valuable for formation evaluation because traditional averaging methods could mask the presence of high-porosity zones within predominantly low-porosity intervals. The frequency distribution approach may preserve information about both the low-porosity and high-porosity components within each depth interval, enabling identification of potential reservoir zones that might otherwise be overlooked. The rightmost upper and lower histograms in FIG. 9 may demonstrate additional examples of predicted distributions that capture complex porosity variability patterns within cutting sample intervals.

The predicted histogram data may be converted into mean log values using a mathematical formula that accounts for the frequency distribution characteristics of each depth interval. The histogram-based mean calculation may be performed using the equation:

$${\overline{x}}_{HIST} = \frac{{\sum }_{i = 1}^{n} {F}_{i} \cdot {V}_{i}}{n} ,$$

where x represents the histogram mean, Fᵢ represents the nth bin frequency value, Vᵢ represents the nth bin central value on the x-axis, and n represents the number of bins. This calculation method may provide mean formation property values that account for the full distribution of properties within each depth interval rather than simple arithmetic averages.

The mathematical approach for computing mean log values from predicted histograms may enable the generation of continuous log curves that represent formation property variations with depth while preserving information about property distribution characteristics within each interval. The histogram-based mean calculation may weight each bin contribution according to its frequency of occurrence, ensuring that the computed mean values accurately reflect the underlying property distribution patterns. This approach may provide more representative mean values compared to traditional averaging methods, particularly for intervals with bimodal or multimodal property distributions.

Advanced statistical analysis may be performed on the predicted frequency distribution histograms to extract additional formation characterization information beyond mean values. Local maxima detection algorithms may be applied to identify whether predicted distributions exhibit unimodal or bimodal characteristics within each depth interval. The local maxima detection process may analyze the frequency distribution patterns to identify peaks that correspond to distinct property value ranges, enabling classification of intervals as having uniform or heterogeneous formation characteristics.

For intervals where bimodal distributions are identified through local maxima detection, separate mean values may be computed for each convolved normal distribution component. The bimodal analysis may decompose complex distributions into individual normal distribution components, enabling calculation of separate statistical parameters for each component. This advanced analysis approach may provide detailed characterization of formation heterogeneity by quantifying the properties of distinct rock types or formation zones within individual cutting sample intervals.

The formation property frequency distributions may include porosity frequency distributions that characterize porosity variability within the cutting sample intervals, providing comprehensive information about reservoir quality and formation characteristics. Statistical parameters may be derived from each predicted porosity frequency distribution to quantify property variability, where the statistical parameters may include mean, standard deviation, and distribution shape characteristics. The statistical parameter extraction may enable quantitative assessment of formation heterogeneity and reservoir quality variations that support drilling decisions and completion design efforts.

The distribution shape analysis may include calculation of skewness and kurtosis parameters that characterize the asymmetry and peakedness of the predicted frequency distributions. Skewness measurements may indicate whether porosity values are predominantly distributed toward higher or lower values within each interval, while kurtosis measurements may characterize the concentration of values around the distribution center. These distribution shape parameters may provide additional geological insights about formation characteristics and depositional environments.

The disclosed methods may be applied to geothermal drilling operations where traditional logging tools may be restricted due to extreme temperature conditions or economic constraints. The drilling cuttings-based approach may provide formation property information in challenging drilling environments where conventional wireline logging may not be practical or cost-effective. The frequency distribution prediction capabilities may enable comprehensive formation evaluation using drilling cuttings as the primary data source, supporting geothermal resource assessment and well completion decisions in high-temperature environments.

The method for automated formation evaluation may include predicting formation property frequency distributions that characterize property variability within cutting sample intervals for new wells by applying a trained machine learning model to consolidated input datasets derived from drilling cuttings photographs of the new wells. The prediction process may utilize the frequency distribution computation methods to generate comprehensive formation property characterization that preserves information about property variability rather than reducing the data to single average values. The consolidated input datasets may be derived from both consolidated rock particles and unconsolidated materials to provide complete formation property predictions for each cutting sample interval.

The methods of well log predictions described herein have been primarily described with reference to wellbore drilling operations. The methods described herein may be used in applications other than in the context of drilling operations for a wellbore. In other embodiments, methods according to the present disclosure may be used outside a wellbore or other downhole environment used for the exploration or production of natural resources.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

1. A method for predicting a target log frequency distribution within a depth interval of a target well, the method comprising:
extracting drilling cuttings from a reference well;
capturing a plurality of photographs of the drilling cuttings, wherein each photograph of the plurality of photographs corresponds to a specific depth interval of a plurality of depth intervals;
obtaining a reference target log from the reference well covering the plurality of depth intervals;
extracting a plurality of image features from each photograph;
capturing property variability within each depth interval including converting the plurality of image features and the reference target log into normalized frequency distributions; and
predicting model property variability by training a prediction model using the normalized frequency distributions of the plurality of image features as input data and the normalized frequency distributions of the reference target log as output data.

2. The method of claim 1, wherein the plurality of photographs comprises white-light illuminated photographs of the drilling cuttings.

3. The method of claim 2, further comprising obtaining ultraviolet illuminated photographs of the drilling cuttings from the reference well and the target well.

4. The method of claim 1, further comprising obtaining auxiliary well logs from the reference well and the target well, wherein the auxiliary well logs are used to train the prediction model.

5. The method of claim 4, wherein the auxiliary well logs comprise gamma-ray logs.

6. The method of claim 1, wherein extracting the plurality of image features comprises segmenting the plurality of photographs to identify individual rock particle features using a residual neural network autoencoder method.

7. The method of claim 6, wherein:
the segmenting the plurality of photographs further identifies unconsolidated material and consolidated rock particles in the plurality of photographs; and
the prediction model comprises a first prediction model for the unconsolidated material and a second prediction model for the unconsolidated material.

8. A system for predicting formation property variability in a target well from drilling cuttings, comprising:
an image acquisition device configured to capture photographs of drilling cuttings samples from wellbore operations;
a processor electrically coupled to the image acquisition device;
a non-transitory computer-readable medium storing instructions that, when executed by the processor, perform a prediction process, comprising:
segmenting the photographs to identify consolidated rock particles and unconsolidated materials;
extracting a plurality of features from the consolidated rock particles and the unconsolidated materials;
converting the plurality of features into normalized frequency distribution histograms that capture property variability within cutting sample intervals;
training, using reference well data, a first prediction model for target consolidated rock particles and a second prediction model for target unconsolidated loose fraction materials, wherein the first prediction model and the second prediction model are configured to predict property variability distributions within the target well; and
applying the first prediction model and the second prediction model to predict formation property frequency distributions that characterize target property variability for the target well.

9. The system of claim 8, wherein the image acquisition device is configured to capture white-light illuminated photographs and ultraviolet illuminated photographs of the drilling cuttings samples.

10. The system of claim 9, wherein the ultraviolet illuminated photographs include fluorescence information for carbonate mineral identification.

11. The system of claim 8, wherein the processor is further configured to obtain auxiliary well logs and incorporate the auxiliary well logs into the training of the first prediction model and the second prediction model and the prediction of the formation property frequency distributions.

12. The system of claim 11, wherein the auxiliary well logs comprise gamma-ray logs.

13. The system of claim 8, wherein extracting the plurality of features comprises using a residual neural network autoencoder method.

14. A method for automated formation evaluation, comprising:
producing drilling cuttings from a reference wellbore;
capturing a photograph of a sample of the drilling cuttings;
analyzing the sample using object-based image analysis on the photograph to segment individual cutting instances and unconsolidated patches;
extracting first image features from each individual cutting instance of the individual cutting instances;
extracting second image features from each unconsolidated patch of the unconsolidated patches using a feature extraction algorithm;
gather feature variability information from the sample by generating frequency distribution histograms for each of the first image features and the second image features;
consolidating the frequency distribution histograms to create input datasets corresponding to specific depth intervals within a target well that preserve the feature variability information;
training a machine learning model using the input datasets and the frequency distribution histograms; and
predicting formation property frequency distributions that characterize property variability within cutting sample intervals for new wells by applying the trained machine learning model to consolidated input datasets derived from drilling cuttings photographs of the new wells.

15. The method of claim 14, wherein the feature extraction algorithm comprises a residual neural network autoencoder method.

16. The method of claim 14, wherein:
the photograph is a white-light illuminated photograph; and
the method further comprises acquiring an ultraviolet illuminated photograph of the sample.

17. The method of claim 16, wherein the ultraviolet illuminated photograph includes fluorescence information for identifying carbonate minerals within the sample.

18. The method of claim 14, further comprising obtaining auxiliary well logs from the reference wellbore and incorporating the auxiliary well logs into the training of the machine learning model.

19. The method of claim 18, wherein the auxiliary well logs comprise gamma-ray logs.

20. The method of claim 14, wherein:
the formation property frequency distributions comprise porosity frequency distributions that characterize porosity variability within the cutting sample intervals; and
the method further comprises deriving statistical parameters from each predicted porosity frequency distribution of the porosity frequency distributions to quantify the property variability, the statistical parameters comprising mean, standard deviation, and distribution shape.
